(19) United States
(12) Patent Application Publication
(10) Pub. No.: US 2004/0029240 A1
(43) Pub. Date: Feb. 12, 2004

(54) Dynamic electroporation apparatus and method

(76) Inventor: Jesse L. Acker, Hackettstown, NJ (US)

Correspondence Address: William Squire, Esq., c/o Carella, Byrne, Bain, Gilfillan,, Cecchi, Stewart & Olstein, 6 Becker Farm Road, Roseland, NJ, 07068, US

(21) Appl. No.: 10/428,612

(22) Filed: May 2, 2003

Related U.S. Application Data
(60) Provisional application No. 60/379,981, filed on May 13, 2002.

(51) Int. Cl. C12N 13/00; C12M 1/42

(52) U.S. Cl. 435/173.6; 435/285.2

(57) ABSTRACT

A housing has a chamber containing a low speed, e.g., 6 rpm, rotating shaft of highly polished stainless steel to avoid bubbles and arcing. A fluid containing cells to be electroporated and molecules associated with the electroporation are introduced into the chamber formed by the gap between the shaft and housing in either batch or continuous flow modes. A capacitor network applies alternating positive and negative pulses of high voltage across the electrodes formed by the housing and shaft to electroporate the cells. After a few pulses, a resistive load is placed across the network to reduce the value of the pulses applied to the electrodes for a relatively longer period than the high voltage pulses to a relatively low harmless level. The relative motion of the shaft electrode and stationary housing ground electrode creates shear stress in the fluid causing the cells to change orientation to expose different cell surfaces to the voltage pulses thereby minimizing cell destruction due to excessive voltage application. The housing is cooled by thermoelectric devices to maintain the temperature of the cells at a safe level.

### FIELD OF THE INVENTION

This application claims the benefit of provisional application serial No. 60/379,981 filed May 13, 2002, entitled “Dynamic Electroporation Apparatus and Method” incorporated by reference herein in its entirety.

This invention relates to an apparatus for the electroporation of cells for inserting selected molecules into the cells and to methods of using the apparatus for insertion of specific molecules into the cells.

### BACKGROUND OF THE INVENTION

This invention relates to electroporation of cells and the insertion of molecules into the cells via the formed electropores.

Electroporation has been used for the insertion of molecules into animal or plant cells from the early 80's. Researchers have demonstrated that short duration, high voltage electrical pulses cause openings to form in the cell membrane through which molecules of interest can be inserted. These openings, referred to as electropores, are regions of increased permeability initiated by local breakdown in the cell membrane caused by high intensity electrical fields. Furthermore, extensive studies by Chang and Chang et al., U.S. Pat. Nos. 4,822,470; 4,970,154; 5,304,486 and Guide to electroporation and Electrofusion, Chang et al. Academic Press, Inc. NY, N.Y. 1992, have shown that these electropores initially propagate below detectable levels of electron microscopy (about 0.2 nm in diameter). After the application of the first electrical pulse, the electropores increase in size over several milliseconds until they reach 60-120 nm in diameter (see FIG. 2). The final size of an opening appears to be limited by the cytoskeleton supporting the membrane.

Much of the early work in electroporation was carried out in static formats. That is, a specialized cuvette containing molded-in electrodes in fluid contact with a fixed volume of target cells and molecules of interest was placed between two electrodes and pulsed with high voltage. Most commercially available electroporation cuvettes are limited in size and can only process small amounts of sample (less than one ml). In static electroporation the percentage of surviving cells containing the molecules of interest seldom exceeds 520% of the original sample. It is difficult to supply adequate cooling to the sample in the static configuration, and many cells are destroyed due to the effects of I²R heating and repeated pulsing on non-moving cells in suspension.

In the 1980's, work was initiated by several researchers on flow electroporation for processing large volumes of cells [references xxx Please identify Nicolau et al, Meserol et al]. Nicolau et al (U.S. Pat. No. 4,752,586, U.S. Pat. No. 5,612,207), Meserol et al (U.S. Pat. No. 6,074,605, U.S. Pat. No. 6,090,617) Flow devices for electroporation generally consisted of parallel electrodes between which the sample flowed. As the electrodes were pulsed with high voltage, it was necessary to continuously cool both the electrodes and the sample undergoing electroporation, as much heat was generated by I²R heating during the extended processing times. The hardware required to implement flow electroporation is relatively complex and expensive.

In summary, static electroporation systems provide a relatively approach to incorporating molecules into cells. However, viable yields of treated cells are limited. Flow electroporation systems were developed to process larger volumes of cells under well-controlled thermal and electrical conditions, enabling improved yields of viable cells. The disadvantages of existing flow electroporation devices are cost and complexity. There is a need in the art for simple, cost effective devices that provide higher yields of viable, successfully treated cells.

### BRIEF SUMMARY OF THE INVENTION

An apparatus according to the present invention is for inserting selected molecules into a cell or other membranous vesicle by electroporation. The apparatus comprises a first electrode; a second electrode forming a sample gap between the first and the second electrodes, means for causing relative movement of the first electrode to the second electrode and means for applying a potential difference between the electrodes in the gap.

In a further aspect, a method and apparatus for electroporating cells includes forming a shear stress in the sample fluid in the gap to cause the cells to change orientation.

In a further aspect, the means for causing the relative movement of the first electrode to the second electrode comprises at least one member selected from the group consisting of a vibration source, a cylinder and piston, a solenoid and a motor.

In a further aspect, the means for causing the movement includes means for causing at least one of relative rotation, translation or vibration between the electrodes.

In another aspect, a method of electroporating a cell comprises introducing the cell into a sample gap disposed between two electrodes, wherein at least one of the two electrode is mobile; and applying a potential difference between the two electrodes while the at least one of the two electrodes is in relative motion thereby electroporating the cell.

In a further aspect, the first electrode is a rotating shaft and the second electrode is a housing receiving the shaft and forming a chamber forming the gap with shaft.

In a further aspect, the means for applying the high voltage pulses includes a switching capacitive network for creating a pulse train of alternating negative and positive first pulses.

In a further aspect, the first pulses have a minimum amplitude of a first value and including means for limiting the amplitude of the following pulses to a given constant value lower than the first value after the expiration of a given period. In a further aspect, the means for applying high voltage pulses includes means for periodically applying successive spaced trains of pulses of exponentially decreasing values to the electrodes.

In a further aspect, the first electrode is a housing forming a chamber in which the gap is disposed, the second electrode being located in the chamber forming the gap with the housing in the chamber, further including cooling means for cooling the housing.

In a further aspect, the cooling means comprises thermoelectric means.

In a further aspect, the means for pumping sample fluid into the gap for one of batch or continuous processing in the gap.

A method of electroporating a target cell according to a further aspect of the present invention comprises introducing the target cell into a sample gap disposed between two electrodes; displacing at least one of the two electrodes; and electroporating the target cell by applying a potential difference between the two electrodes during the displacing.

In a further aspect, the applying the potential difference includes applying voltage pulses of alternating polarity at spaced intervals.

In a further aspect, the spaced intervals each have a first time value and the pulses have a pulse width of a second time value, each interval first value being at least three times the magnitude of the second value.

In a further aspect, the applying the potential difference includes applying a plurality of spaced voltage pulses, at least one of the voltage pulses being followed by a first time period encompassing the time of occurrence of the at least one voltage pulse, no pulses being applied in the first time period.

In a further aspect, the applying of the pulses includes forming a network of capacitors and providing means for discharging the capacitors through the sample to produce the pulses, the means for discharging for causing the pulses to exhibit an exponential decaying amplitude from a first value, the method including reducing the voltage amplitude of the first value across the capacitors to a substantially lower second value for a second time period following the generation of the pulses of the first value.

In a further aspect, the forming the second values of the pulses at a substantially constant amplitude.

In a further aspect, the method includes providing a target cell comprising at least one of a suspended cell, an adherent cell or a cell ghost.

In a further aspect the method further comprises introducing a molecule of interest into the sample gap.

In a further aspect, the applied potential difference comprises voltage pulses of alternating polarity spaced at intervals each of which is at least three times the width of the voltage pulses.

In a further aspect, one or more voltage pulses are followed by a period encompassing the time of one or more voltage pulses where no pulses are applied to the two electrodes and where a resistive load is shunted across the sample cell to reduced the voltage across the capacitor source to a relatively low value as compared to the applied potential difference prior to reconnecting the load.

In a further aspect, in such methods, the target cell may be one of a suspended cell, an adherent cell or a cell ghost, especially wherein a molecule of interest is introduced into the sample gap. In a preferred embodiment, the molecule of interest is selected from the group consisting of proteins, peptides, nucleic acids, microparticles, nanoparticles, polymers, conjugated molecules and labeled molecules.

Preferably, the molecule of interest is selected from the group consisting of drugs, therapeutic candidates, members of a candidate library, ligands, receptors and optically detectable molecules.

In an additional aspect, the molecule of interest is optically detected in the target cell.

In accordance with a further aspect, the optical detection comprises means including at least one of spectrophotometry, imaging, microscopy, laser scanning, fluorescence, flow cytometry or cell sorting.

In a further aspect, the molecule of interest is selected from the group consisting of proteins, peptides, nucleic acids, microparticles, nanoparticles, polymers, conjugated molecules, labeled molecules, drugs, therapeutic candidates, members of a candidate library, ligands, receptors and optically detectable molecules.

### DEFINITIONS

As used herein, and unless specifically stated otherwise, each of the following terms has the indicated meaning.

“Cell,” when used in reference to the target of electroporation, means a biological or synthetic cell, microvessel or vesicle including, but not limited to, a biological cell of plant, animal, protist, fungal or moneran origin or a cell ghost, microvesicle, microcapsule or microcompartment. Cells may be living or nonliving, suspended or adherent, and naturally occurring or recombinant. They may be biologically produced, or they may be manufactured, processed, modified or engineered by synthetic or nonbiological means.

“Electroporation” means the formation of a functional opening in a cell membrane, wall or layer or boundary caused by exposing the cell to an applied external electric field, optionally followed by passive diffusion or electrophoretic transfer of molecules into the cell.

“Electropore” refers to a functional opening in a membrane, wall or layer or boundary, particularly a cell membrane or cell wall.

“Free tumbling zone,” when used in reference to a flow electroporation device, means a region where the shear velocity is relatively constant across the cross-section of flow.

“I²R heating” refers to resistive heating described by the classical Joule heating equation involving current squared multiplied by resistance.

“Mobile,” when used in reference to an electrode of a device, means the electrode is capable of movement relative to a second electrode of the device in a manner that contributes to the utility of the device.

“Molecule,” when used in reference to electroporation of a molecule into a cell, means and includes any molecule, conjugate, molecular complex, substance or particle that can be internalized by electroporation, including conjugated, immobilized, dyed, tagged and/or labeled counterparts.

“Poration” means electroporation.

“Rotary” refers to a method or device relying on use of a rotor or rotational motion.

“Rotational,” when used in reference to an electroporation device, refers to the motion of a device component as distinct from the rotational movement of target cells.

“Target cell” refers to a cell subjected to electroporation.

“Treated cell,” when used in reference to a target cell, means a target cell into which a detectable amount of molecule or particle of interest has been successfully electroporated.

### BRIEF DESCRIPTION OF THE DRAWINGS

FIG. 1 is an exploded view of an electroporation apparatus according to an embodiment of the present invention;

FIG. 2 is an isometric view of the apparatus of FIG. 1 when assembled;

FIG. 3 is a side elevation view of the apparatus of FIG. 2;

FIG. 4 is a sectional elevation view of the apparatus of FIG. 3 taken along lines 4-4;

FIG. 5 is a schematic circuit diagram of a high voltage switching network for producing the waveform of FIG. 8 across the electrodes of the apparatus of FIGS. 1-4;

FIG. 6 is a longitudinal sectional elevation view of the apparatus of FIGS. 2 and 3;

FIG. 7 is a waveform diagram of the output of a capacitive discharge circuit;

FIGS. 8 and 9 are a waveform diagrams employing the capacitive discharge circuit of FIG. 5 with different parameters and are two embodiments among several that can be used to effectively electroporate a cell in suspension, FIG. 8 shows the waveforms produced from the capacitor bank of FIG. 5 charged up to a high voltage (here, between about 370 v to 740 v), wherein high voltage, high current switches intermittently connect the capacitor bank to the sample electrodes;

The pulse formats of FIGS. 8 and 9 are two embodiments among several that can be used to effectively electroporate a cell suspension. FIG. 8 shows the time relationships between the voltage format and the pore formation.

FIG. 10 is a block schematic diagram of a circuit for operating the apparatus of FIGS. 1-4.

FIG. 11 shows a voltage waveform for the 740 volt experiments of Example 1 for turkey cells.

FIG. 12 shows a current waveform for the experiments of Example 1.

FIG. 13 shows an impedance waveform for the experiments of Example 1.

FIG. 14 shows a low voltage impedance plot for non-electroporated cells.

### DETAILED DESCRIPTION OF THE INVENTION

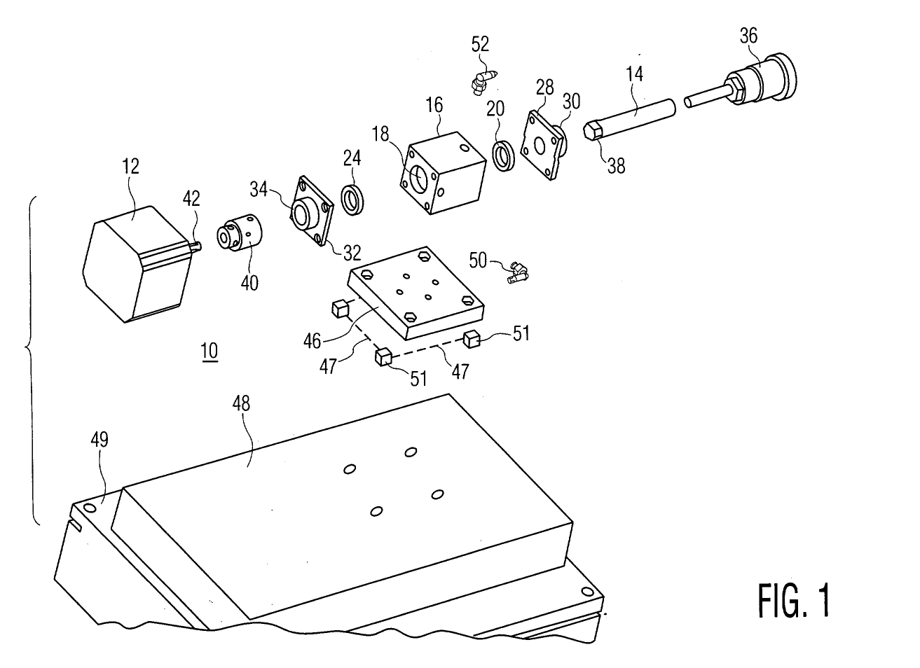

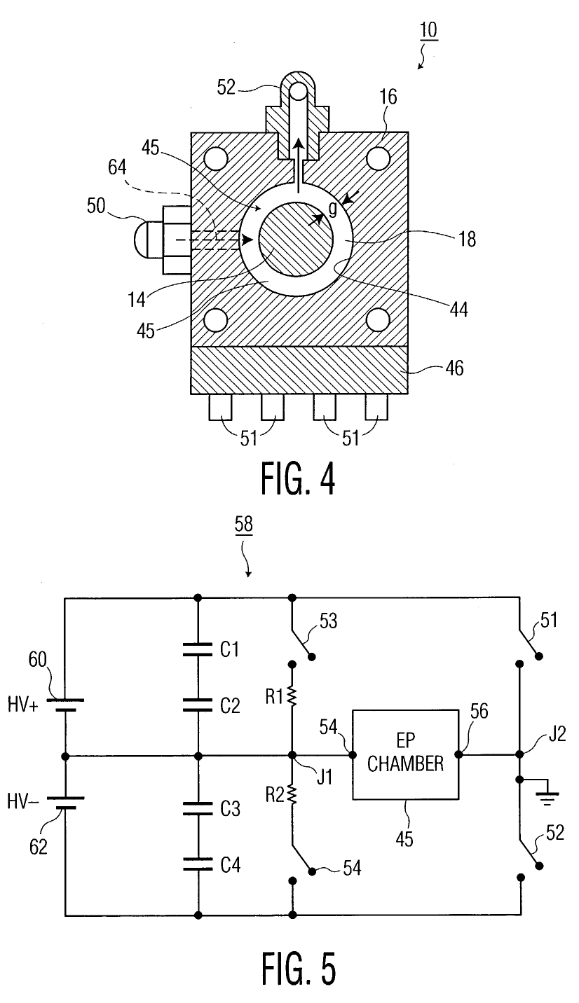

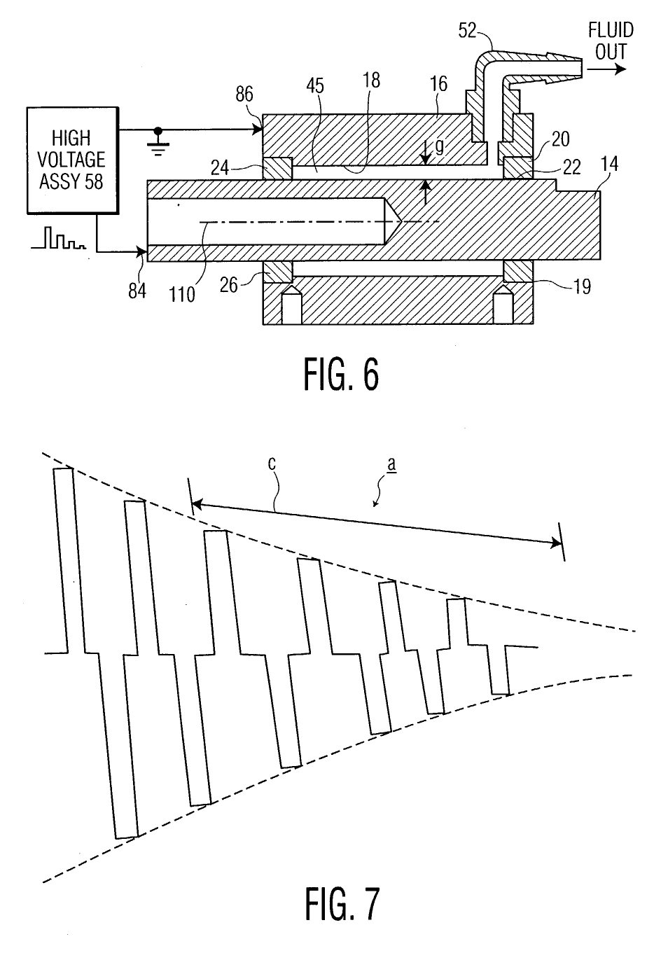

The apparatus according to an embodiment of the present invention provides a simple, cost effective apparatus and method for electroporating a wide variety of cells with high yields of viable, treated cells over a wide range of sample volumes and experimental conditions. In the embodiment of FIG. 1, apparatus 10 comprises a drive motor 12 for driving shaft 14 which forms an inner electrode of the apparatus 10. Housing 16 has a longitudinal circular cylindrical bore 18, FIGS. 4 and 6. A conventional annular ring fluid seal 20 seals the shaft bore 18 at one bore end 19 and is in recess 22 in the housing in communication with the bore 18 at the one bore end. The seal 20 permits the shaft to rotate relative to the seal and act as a bearing for the shaft 14. The seal 20, for example, may be rubber, synthetic rubber, teflon, Delrin, spring actuated rubber or synthetic seals. Seal 24, which may be identical to seal 20, is in recess 26 at the other end of the bore 18. The seals 20 and 24 are dielectric and serve to electrically isolate the shaft 14 from the housing 18.

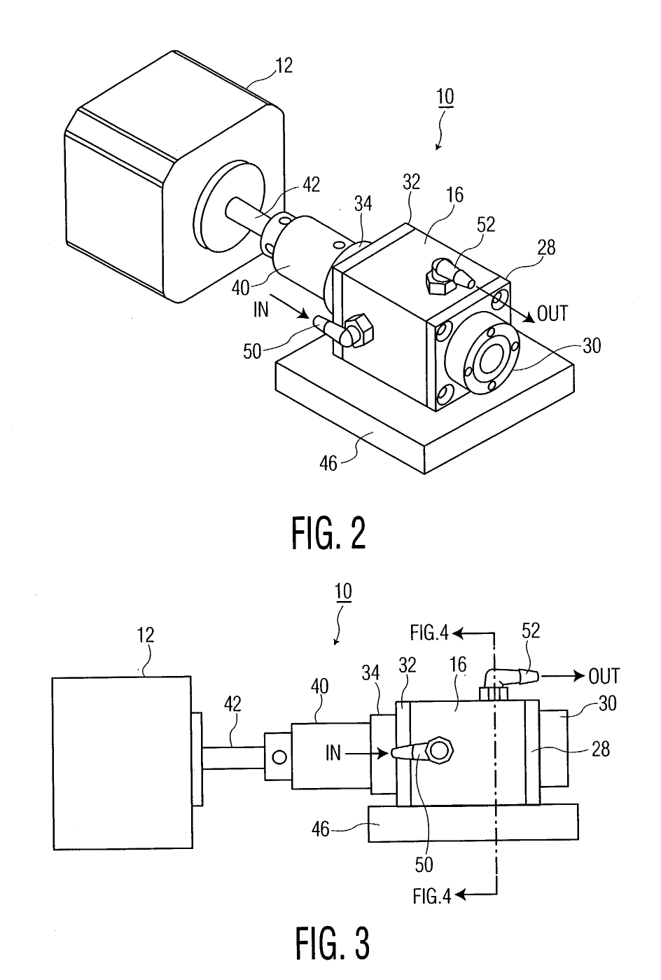

In FIGS. 1, 2 and 3, an end plate 28 is bolted to the housing 16 capturing the seal 20 to the recess 20. The end plate 28 has a collar 30 through which the shaft 14 passes. A second end plate 32 is secured to the housing other end to capture the seal 24 to the recess 26. The end plate 32 has a collar 34. The shaft 14, FIG. 1, has an internal bore 110 which serves as a receptacle for receiving a high voltage plug which itself has an internal bore for receiving a high voltage from a heavy supply wire terminated in a cylindrical close fitting plug (not shown) The end plate 32 has a collar 34 and is fabricated from a dielectric material which electrically isolates shaft 14 from body 16.

The shaft end 38, FIG. 1, is connected to coupler 40. Coupler 40 fabricated of dielectric material, secures the shaft 14 to the motor 12 drive shaft 42 in electrical isolation thereto. Thus, the shaft 14 is electrically isolated from the motor 12 and shaft 42 and from the housing 18 and its components by coupler 40 and via the dielectric seals 20 and 24. The housing bore 18 wall 44, FIG. 4, is concentric with the shaft 14 throughout and are spaced apart to form a chamber 45 defined by gap g between the housing 16 and shaft 14. The gap g is about 2 mm between the wall 44 and shaft 14. The gap g and its length in the housing 18 along the shaft 14 has a volume of about 1.5 cc.

The housing 16 is mounted on a metal plate 46, FIG. 1, which in turn is mounted on a cooling metal plate 48 which is a relatively large mass for rapidly cooling the housing 18. The plates 46 and 48 are highly thermal conductive and have a relatively high thermal mass to maintain the housing temperature within the desired temperature range.

In FIGS. 1-4 and 6, a fluid inlet nipple 50 is in fluid communication with the chamber 45 adjacent to end plate 32 for flowing fluid into the chamber to be processed by the apparatus 10. A fluid outlet nipple 52 is coupled to chamber 45 adjacent to the end plate 28 for receiving the processed fluid from the chamber 45. Suitable conduits are connected to the nipples 50 and 52 to carry the fluids to and from the chamber 45. The shaft 14 is preferably rotatably driven about 5-6 rpm.

A first electrical conductor (not shown) is connected to the housing forming an electrode and a second electrical conductor is connected to the shaft 14 which forms a second electrode, and connected for example, high voltage connector 36, FIG. 1. The electrodes are for applying a pulsed high voltage across the chamber 45 gap g. The shaft 14 serves as one electrode for the high voltage and the housing serves as the other electrode across the gap g, FIG. 6. The first and second electrodes are schematically shown in the circuit diagram of FIG. 5.

The switching circuit 58 of FIG. 5 generates the pulsed high voltage applied across the chamber 45. In FIG. 5, the circuit 58 comprises a capacitive network of two capacitors C1 and C2 connected in series across the positive high voltage source 60 and across the series network of switch S3 and resistor R1. Two series connected capacitors C3 and C4 are connected across the negative high voltage source 62, the sources 60 and 62 being about the same value, e.g., 1000 volts. The capacitors C3 and C4 are connected across the series network of switch S4 and resistor R2. The chamber formed by housing 16 and shaft 14 is connected between the junction J1 of resistors R1 and R2 and the junction J2 of switches S1 and S2 which are connected in series across the high voltages 60 and 62. The switches S1, S2, S3 and S4 are computer controlled.

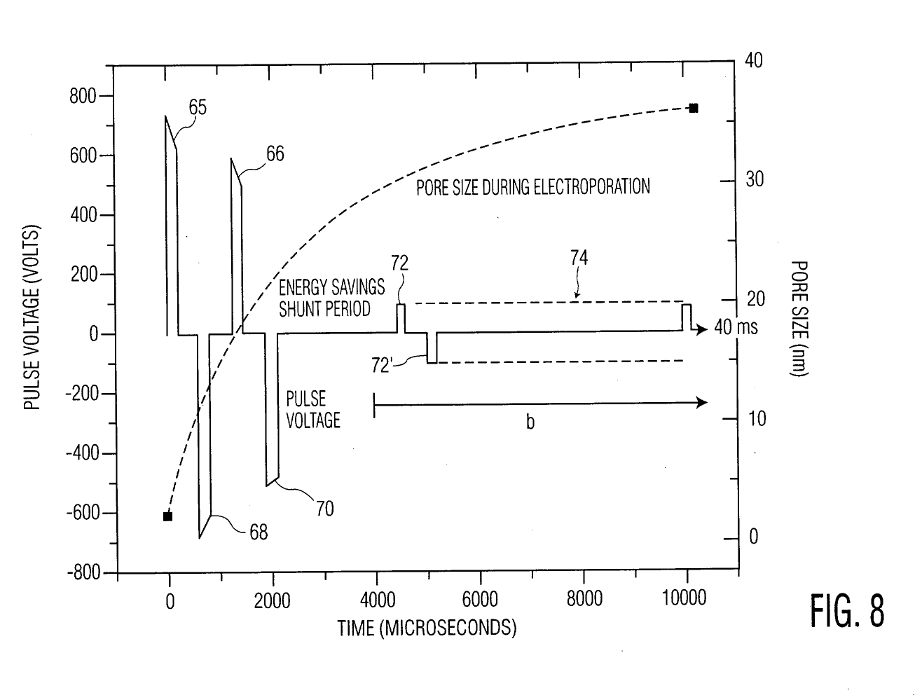

The alternate closing and opening of switches S1 and S2 discharges their respective capacitor networks across the housing 16 chamber 45 creating high voltage positive pulses 64 and 66, FIG. 8, and high voltage negative pulses 68 and 70. After these pulses are created, the switches S3 and S4 are simultaneously closed providing a rapid decay of the pulse train in region b. This region may be 2.4 milliseconds. The following lower voltage pulses prevent the sample suspension from over heating and destroying the target cells molecules that are being processed in the chamber 45, which over heating would otherwise occur in the presence of the high voltage pulses which may have a peak value of about 700 volts.

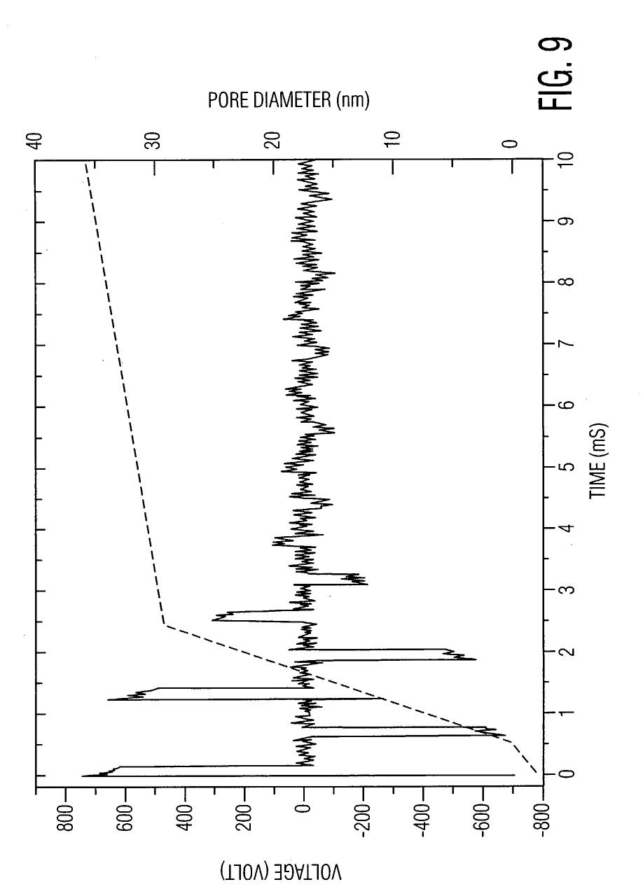

The high voltage pulses created prior to the closing of switches S3 and S4 promote the formation of the electropores on the cell membrane. The lower voltage pulses resulting from the rapid decay of voltage across capacitors C1, C2, C3 and C4 during the period when S3 and S4 are closed, function primarily to electropherese the molecules of interest in the sample suspension through the now-opened pores. FIG. 9 shows another embodiment of pulses for forming electroporation of the molecules of interest.

The system utilizes coaxial cylinders with the sample to be electroporated filling the volume 45 between the two cylinders, which are insulated electrically from each other. Voltage is applied across the sample through electrical connections to the two cylinders from a high voltage pulsed power supply. One of the cylinders shaft 14 rotates with respect to the other. The rotational rate is adjusted to optimize conditions of cell orientation during electroporation while maintaining effective cooling.

In other embodiments, translational (e.g., reciprocal) or vibrational motion, for example, may be used to vary the time-dependent position of one electrode, e.g., the shaft 14, relative to the other, e.g., the housing 16. Uniform, accelerated, continuous or intermittent motion can be used to move one or both electrodes. In designs relying on the motion of two (or more) mobile electrodes, the electrodes may move simultaneously or sequentially.

The present invention is based on the discovery that when one or both electrodes rotate, the shearing of the fluid layers in the chamber 45 creates a tumbling effect on the target cells of interest in which the cell surfaces are continually in motion and continually changing orientation. This has the advantage of obtaining high yields of electroporated cells due in part to the ability to create randomly rotating cell conditions. For any particular flow cell configuration, the flow dynamics can be optimized to develop a region of the flow where most of the cells are ‘free tumbling’. This region exists where the shear velocity is relatively constant across the cross-section of flow. As the cells change orientation in random fashion due to the relative motion of the shaft to the housing which causes the cell-carrying-fluid to be in motion, increasing the probability of developing additional electropores increases while avoiding destruction from repeated pulsing into the already opened electropores in the same cell walls. When cells are in ‘free tumbling’ they continually turn new surfaces to the electrical initiating source, the high voltage pulses avoiding repeated pulsing into already opened pores and possible destruction of the molecules or cells. This electroporation of the cells creates pores in their surfaces which enables maximum transfer rates of material into cells with minimum cell loss. Yields as high as 85% have been obtained.

In one embodiment, one electrode, the shaft 14, is slowly rotated to produce a relatively flat shear velocity profile across the gap g, encouraging random free tumbling of the cells to be electroporated. In random free tumbling during electrical pulsing, different areas of the cell surface are exposed to the pulsed electric field as the cell changes orientation having the effect of producing a more uniform distribution of electropores.

Another embodiment is to counter-rotate the inner and outer electrodes to produce additional shear in the fluid sample between the electrodes.

The sample consisting of cells, buffer and molecules to be electroporated into the cells, is introduced into the gap g between the electrodes, the housing 16 and shaft 14, via fluid fittings, nipples 50 and 52. Rotation and electroporation ensue for a number of pulse trains (One pulse train is defined as a predetermined series of voltage pulses starting with several high voltage pulses spaced several hundred microseconds apart, followed by a longer series of lower voltage pulses lasting approximately 20 to 40 milliseconds. Examples of useful pulse trains are shown in FIGS. 8 and 9.) Post electroporation, the sample may be expelled with a buffer and/or another fresh sample, into a collection vessel (not shown).

Another embodiment is to flow a sample continuously through the apparatus 10 at a low enough flow rate that ensures sufficient pulses to effect adequate electrical transfer of the molecules into the target cells. This may also have the desired effect of enhancing the random tumbling of the cells.

The fluid dynamics of the rotary system is superior to that of linear flow electroporation of the prior art, as the number of boundary layers is reduced by one or two, depending upon whether one or both electrodes rotate. Boundary layers trap cells that may then be subjected to excessive pulses and may be destroyed.

The rotational speeds can be low (e.g., as low as 0.1 revs/sec) since a near constant shear velocity pattern across as much of the gap g between the electrodes is desired. Shear is produced by the relative rotational velocity of the two cylindrical electrodes formed by the housing 16 and shaft 10 and the viscous drag of the fluid sample in the chamber 45 relative to the two cylindrical surfaces. Higher shear velocities may encourage laminar flow, which tends to keep the cells in one alignment along a lamina layer, which is not as desirable as random tumbling.

In operation, sample is pumped into the gap g through fluid fitting bore 64, FIG. 4, via nipple 50 by a pump (not shown). Shaft 14 is slowly turned by a stepper motor 12 under control of a controller at such a rate to produce a desired shear velocity profile (e.g., to encourage ‘free tumbling’ of the sample cells in gap g). After gap g has been filled, Shaft 14 is turned. While the shaft 14 is turning, high voltage pulse trains are applied between the shaft 14 and housing 16. Depending upon the dimensions, shape and membrane breakdown potential of the target cells, starting voltage gradients between about 1.85 kv/cm and 3.7 kv/cm may be applied to the sample in the gap g. For a gap of 2 mm, the corresponding voltage range would be about 370 v to 740 v. Pulses may take the forms illustrated in FIGS. 7-9. As these high voltage, high current, e.g., 100 amperes, pulses are advantageously be produced by capacitor discharge via the circuit 58, FIG. 5, they usually have an exponentially decaying envelope as shown

A series of pulse trains, several examples of which are depicted in FIGS. 7, 8 and 9, can be effective in the electroporation of molecules into target cells. There is a theoretical relationship between electropore formation and electrical gradients surrounding the cells. Short, high intensity pulses (about 1.75 kv/cm-3.7 kv/cm) are applied to create electropores, which subsequently grow in size up to a diameter of 60-120 nm. At a time after the initial pore formation when the pores have grown to about 40-60 nm in diameter, a low level electric field is applied to cause electrophoresis of molecules into the target cells through the pores. This low field intensity (approx. 0.75 kv/cm) is maintained for about 38 ms and then shut off. Molecules in solution migrate into cells under the influence of the low level electric field as well as by diffusion. However, the diffusion rates are but a small fraction (e.g., one twentieth) of the electric field driven rates.

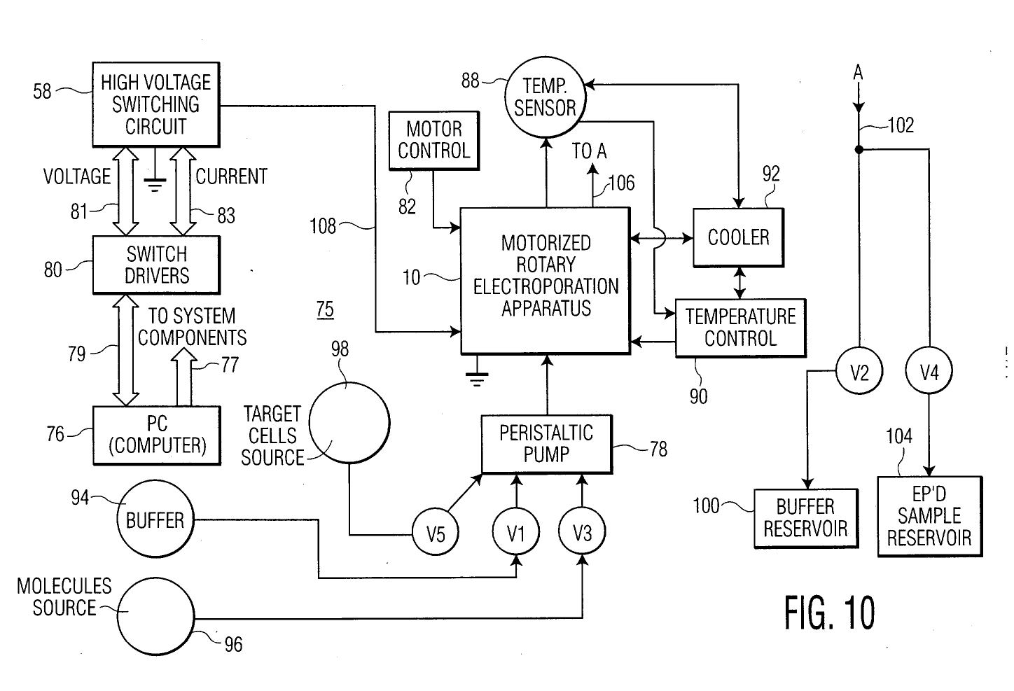

The first high voltage pulses initiate electropore formation in the cell membrane facing the cathodic (negative) electrode. (In one embodiment, sequentially reversing polarity of the electrical pulses is used to create electrical stress on the reverse side of the cell, encouraging the formation of additional electropores.) During the following pulse intervals, the voltage amplitude is decreased rapidly as the electropores increase in size. After this interval, a train of low voltage pulses enables electrophoresis of molecules through the opened pores in the cell membrane. After about 64 low voltage pulses, the voltage is turned off by a controller as will be described below in connection with FIG. 10. From this point, the pores reseal and additional pulse trains are applied. After completion of the required pulse trains, the cells are removed for analysis, subsequent processing or use.

The waveforms of FIGS. 7, 8 and 9 are produced from a capacitor bank circuit 58, FIG. 5, charged up to a high voltage (between about 370 v to 740 v). High voltage, high current switches S1 S4 and S2 intermittently connect the capacitor bank to the sample electrodes, the housing 16 and shaft 14. As current flows into the gap g during electroporation, the capacitor voltage decays exponentially causing the pulse amplitude to follow suit. Heating of the sample from the high currents is an ever present danger that is favorably addressed by the efficient heat removal properties designed into the preferred apparatus 10 of the present invention. In the preferred embodiment, after the first few pulses 64, 66, 68 and 70, FIG. 8, which are involved in early pore formation, resistors R1 and R2 are shunted across the capacitor bank for several pulses, dissipating much of the energy as heat in the resistor. The capacitor voltage of the capacitors C1-C4 drops rapidly during this shunting period. After this period, the shunt is removed and the lower constant value voltage pulses are applied across the gap. At this point, electropores formed are increasing in size to about 40 nm and higher and are receptive to the ingress of molecular material into the cells through their disrupted membranes. FIG. 8 shows the time relationships between the voltage format and the pore formation as described. This pore formation period lasts about 38 ms, after which the switching ceases and the capacitor bank charges back up fully to be ready for the next pulse train, while the electropores reseal between one pulse train and the next.

The pulse formats of FIGS. 7, 8 and 9 are three embodiments among several that can be used to effectively electroporate a cell suspension. The waveform of FIG. 8 is a preferred embodiment. The waveforms of FIGS. 7, 8 and 9 are produced by the circuit 58 of FIG. 5. The outer electrode, the housing 16, of the apparatus 10 is at ground level, two separate high voltage supplies 60 and 62 are used to formulate reversing polarity pulse trains.

When the system is first turned on, the capacitors C1 through C4 charge up to the voltages of the plus and minus supplies. Time to charge to maximum voltage is less than three seconds. The switches S1 through S4 are controlled by switching logic advantageously driven by a computer 76 in the system 75 of FIG. 10 to be described below. The waveforms of FIGS. 8 and 9 are an example of waveforms generated by the switching capacitive pulse generating and discharge circuit 58, FIG. 5. Initially, switch S1 is closed for about 175 μs, applying a positive, high voltage pulse to electrode 54, FIG. 5 (the rotating shaft 14) with respect to the housing 16, which is held at ground potential. After 175 μs, generating pulse 65, both switches S1 and S2 are open for approximately 450 μs. Switch S2 is then closed, applying a negative pulse voltage 68 on the shaft 14 with respect to the housing 16 at ground potential. After 450 μs, the cycle is repeated, generating positive pulse 66 and negative pulse 70 in succession. Following the fourth negative pulse 70, switches S3 and S4 are closed for about four pulses and then opened. This discharges the capacitors C1-C4 to a low level potential. This minimizes heating the cells in the gap 6 with multiple high energy pulses as might occur in the pulse waveform a of FIG. 7, period c, if the pulse train generated would be permitted to decay naturally in time, which would occur without the discharge resistance network of resistors R1 and R2, FIG. 5, in use. The high voltage pulses in period c of FIG. 7 might generate undesirable heat and possibly damage the cells prior to completion of the electroporation process, and thus this embodiment is least desirable, but might be appropriate for certain implementations.

Switches S1 and S2 are then alternately closed and opened approximately 32 times in period b, FIG. 8, generating the low voltage pulse train 74 of slowly decreasing amplitude value in this period, pulses 72 and 72′. After period b, all switches are opened and the capacitor bank charges up to the supply voltage to be ready for the generation of the next pulse train. Switches S3 and S4, are designated ‘energy reduction switches,’ as they are used to absorb energy by shunting the capacitor bank with a low resistances R1 and R2 to discharge the capacitors. This prevents the overheating of cells by excessive exposure to high energy pulses in region c, FIG. 7. During this period, the load switches S1 and S2 are left open.

When the capacitor voltage has dropped to about 150 v, the load voltage switches S1 and S2, are then actuated sequentially and the switches S3 and S4 are opened, putting low voltage reversing polarity pulses across the chamber 45, FIG. 6. As the pores have opened almost as far as they are going to, the low voltage pulses act in period b, FIG. 8, to transfer molecules of interest into the cell pores via electrophoresis. The waveform of FIG. 8 results from this process.

FIG. 7 can be derived by just alternating the switches S1 and S2 until the capacitor voltage has essentially gone to zero. This is a pure exponential discharge. FIG. 9 is similar to that of FIG. 8 except that the load switches S1 and S2 are operated during the energy reduction phase, resulting in more heating of the sample in the chamber 45.

The embodiment of FIG. 8 is particularly effective, as negligible or no energy goes to the load while the pores are opening up. Among FIGS. 7-9, FIG. 8 represents the most energy conservative case with the least heating of the sample in the sample gap g.

As many as eight pulse trains or more have produced as much as 85% yield of viable cells containing detectable amounts of the molecules of interest. A multiplicity of pulse trains is usually necessary, as the first pulse train may create electropores in only a small percentage of the cell population. Multiples may be needed not only to add to the number of successfully treated cells, but also to have additional sides of the same cells porated as they rotate randomly. This is made possible, as the almost three seconds between pulse trains is sufficient time for the earlier formed pores to reseal and for new entry pores to be formed.

Three turns of the shaft 14 are usually adequate to accomplish thorough electroporation of the sample as during the time of rotation as many as eight pulse trains can be applied to the sample in gap g. It is believed that the tumbling of the cells occurs in response to the rotation of the shaft 14 due to the molecular attraction of the fluid of the sample in the chamber to the shaft. Thus the fluid is placed into motion by the rotation of the shaft 14. The rotation of the shaft also is believed to possibly cause some rotation of the fluid which also exhibits some shear velocity gradients' due to this rotation. This gradient causes the cells in the fluid sample to change orientation so as to expose different cell surfaces to the high energy pulses that are applied during the rotation of the shaft 14 and thus prevent overheating individual cells which might destroy them.

The design of the heat transfer feature of the apparatus 10, FIGS. 1-4, is more efficient than prior static or flow electroporation systems. The negative electrode is the outer housing 16, and, as noted above, is at ground potential. The outer housing 16 is mechanically, thermally and electrically attached to the cooling plate 48, FIG. 1, and plate 46 by an array of intermediate thermoelectric cooling devices 51, FIG. 4, at the interface with plates 46 and 48. The array is represented by dashed lines 47.

The devices 51 may have temperatures of about −10° C. to −6° C. This tends to keep the housing 16 at the desired cooled temperature to maintain the sample cell temperature in chamber 45 in the safe region. The electroporation chamber 45 is thus encased by a cooled surface, the housing 16, causing the chamber 45 to be thermally isolated from the ambient environment.

Furthermore, the rotation of the shaft 14 acts to diminish the static boundary layer of fluid at the shaft surface, due to the molecular attraction of the fluid to the shaft, and subsequent shearing of the fluid layers near the surface and resulting movement thereof increasing heat transfer from the sample fluid to the housing 16 during electroporation. That is, shear velocity may be introduced in the fluid at the boundaries with the rotating shaft 14. Thus the red blood cells change orientation in the presence of this shear velocity. Prior art systems that flow fluid through the sample chamber typically do this with laminar flow which causes the cell surfaces to maintain a constant orientation and face generally in one direction. This constant orientation unduly over exposes the same cell surfaces to repetitive high energy pulses and thus overheat or destroy the cells.

Preferably, the sample in chamber 45 exhibits a flow rate of about 0.33 cc/min. which may be in the range of about 0.5 to 0.05 cc/sec. This flow rate is based on flow dynamics which may be determined by the viscosity of the sample, the chamber dimensions and surface conditions, and aerodynamics and shape of the cells being processed. In addition, the pulse characteristics are also determined according to cell characteristics. By providing rotation of the shaft electrode, slower flow rates through the chamber 45 than those used in the prior art are employed, but in combination with cooling provide superior yields.

By pulsing the cells as disclosed herein, exposing them to the low level orientation change by rotation of the shaft 14, the 2 mm gap g, at about 4-5 ohms resistance between the shaft 14 and housing 16 in the presence of a 700 volt pulse could create excessive heat and arcs, i.e., 100 amp pulse currents. The target cells may only be raised in temperature to a maximum safe temperature of about 42° C. The fluid in chamber 45 may exhibit a 10-20° C. rise in temperature due to the pulsing and, therefor, requires the cooling to preclude the cells from overheating.

In the prior art systems, no cooling is provided and undesirable boiling of cells may occur employing a static electroporation device. A high current pulse may create a pressure pulse and wave from an arc and literally cause an explosion resulting from excessive gas evolution.

Heat removal during electroporation is important both in keeping the cells at safe temperatures and in preventing the initiation of arcs under high voltage pulsing conditions. Electroporation buffers are usually comprised of low impedance solutions to establish the best conditions for electropore formation and ion migration. With any low impedance buffer during electroporation, electrolysis occurs, causing the formation of bubbles at the electrode surfaces. If the surfaces are rough or the temperatures are high during high current phases, large bubbles may form. Breakdown can occur across the meniscus between large bubbles, initiating the propagation of an arc. Arcing may be quite dramatic, with high gas pressure formation resulting. If these pressures are not dissipated by the elastic deformation of the chamber walls, rupture may ensue. (The invention of Meserol et al. U.S. Pat. No. 6,074,605 teaches the use of silicon rubber chambers to absorb pressure shock). To avoid this occurrence, the chamber 45 walls have a fine finish to keep bubble size small and to be very well cooled to prevent the formation of large bubbles and arc initiation. As a result, the surfaces of shaft 14 and the housing 14 forming chamber 45 are highly polished to a fine surface finish. Highly polished surfaces also avoid protein build up. Additionally, the rapidly reversing of the polarity of the applied voltage pulses in the apparatus 10 may act to quench arcs in formation, thus preventing propagation of arcs which are destructive of the cells.

Chromium nitride materials for coating the shaft and housing surfaces in the chamber 45 are costly. In this embodiment, the housing 16 and shaft 14 preferably comprise 303 stainless steel which avoids the problems with other materials in the shaft and housing.

Instead of elastic walls as in the prior art, a more rugged construction and improved heat transfer of the present apparatus prevents the buildup of excessive temperatures within the electroporation chamber 45. The apparatus 10 includes a configuration in which the housing 16 or ground electrode forms part of the cold plate of the thermoelectric cooler attached to the housing 16. Since the ground electrode completely surrounds the sample, it acts to insulate the sample from the external environment and exposes the sample to the cold surface of the cooler.

In FIG. 10, system 75 includes a PC computer 76 coupled to high voltage switching circuit 58. The output of circuit 58 is applied to the electroporation apparatus 10 with the high voltage terminal 84, FIG. 6, connected to the high voltage output of circuit 58 and to the shaft 14. The circuit 58 ground terminal 86 is connected to the housing 18. Motor control 82 is connected to the shaft 14 for rotation of the shaft, FIG. 10.

A temperature sensor 88 which may include an A/D converter for providing a digital output, is coupled to the housing of apparatus 10 for measuring the housing temperature. The sensor 88 is also coupled to the cooler devices 51 represented by cooler 92 for sensing the cooler 92 and housing 16 of apparatus 10 temperature. Control 90 controls the cooler 92 and housing 16 temperature based on the sensed housing temperature. Pump 78 receives buffer fluid solution from buffer 94 supplied to the apparatus chamber 45 via valve V1. The pump also receives the molecules 96 to be inserted into the electroporated cells from source 98 via valve V3. Target cell source 98 is applied to pump 78 via valve V5. The various valves may be controlled by the computer 76. A buffer reservoir 100 forming a chamber cleaning wash receives the buffer fluid via valve V2 which is supplied after the valve V4 is closed and after the sample electroporated and with the inserted molecules is received in reservoir 102 via valve V4 from line 102. At this time the valve V2 is closed and valve V4 is open. The electroporated sample reservoir 104 reservoir receives the electroporated and molecule inserted sample from input line 102 via valve V4.

In operation, in a batch static process, computer 76 includes a computer program (not shown), FIG. 10, for operation of the high voltage switching circuit 58 and other components of the system 75 via bus 77. Pump 78, which is operated by the computer via bus 77, transfers the correct amount of cell sample from source 98 and molecules 96 to be inserted into the electroporated cells into the chamber 45 gap g (FIG. 6) to fill the chamber 45. The pump 78 is stopped by the computer via bus 77 and the rotor shaft 14 is then rotated by control 82 under control of the computer 76 via bus 77.

A set pattern of electrical pulses as described above are applied from switching circuit 58 to apparatus 10 via line 108. The pulses are applied by the circuit 58 under control of the computer 76. The computer 76 applies control signals to circuit 58 via bus 79, switch driver 80 and busses 81, 83. After the requisite number of electrical pulses as described in connection with FIGS. 7-9, electroporation ceases under control of the computer 76. The pump 78 is now activated by the computer 76. The pump 78 transfers the electroporated sample from apparatus 10 to a collection vessel reservoir 104 via output line 106 to input line 102 with the now opened valve V4. This transfer of the sample is performed by inputting a controlled amount of new fluid either from the cell source 98 and molecules from source 96 or buffer source 94.

The valve V2 is closed at this time and the valve V4 is open, under control of the computer 76. When the electroporated sample has been cleared from the apparatus 10, V4 closes. This action replaces the chamber 45 contents with a new sample from sources 78 and 96 or with buffer from source 94. If with a new sample, the electroporation process can resume. If it is replaced with buffer, then sufficient buffer will be run through apparatus 10 to completely wash the apparatus through Valve V2 into reservoir 100 after which a new sample can be pushed through to replace the buffer and the process resumed. The process starts when a new mixture of target cells and molecules is pumped into a clean dry apparatus 10. After electroporation is complete, the electroporated sample must be pumped into the sample reservoir by virtue of displacing it out of apparatus 10 by either buffer or by more of the same sample mixture. If by buffer, then when the electroporated sample plus a little buffer has passed into the reservoir 104 as measured by the number of turns of the peristaltic pump (which is a positive displacement pump), V4 is shut off, V2 is opened and sufficient amounts of buffer is passed through apparatus 10 to clean it of the previous sample. The next sample can be used to displace the buffer resident in apparatus 10 into reservoir 100, after which V2 is closed and EP ensues.

Another embodiment employs a continuous process rather than a batch process as described above. In the continuous process, larger volumes of sample are processed. In this process, a sample comprising cells from source 98 and molecules from source 96 is flowed continuously into and out of the chamber 45 at a rate that allows a unit volume entering the chamber 45 to receive an adequate number of pulses to achieve acceptable electroporation results prior to exiting the chamber 45. This procedure is performed by the system 75 wherein the pulses applied by circuit 58, motor control 82 and valves VI-V5 and pump 78 are all operated automatically under control of the computer 76 via bus 77 (and other lines not shown).

The computer 76 is programmed to operate the motor control 82, pump 78, and open and close the valves accordingly, as well as rotate the shaft 14 of apparatus 10 and apply the high voltage pulses from circuit 58 in the proper sequence. In each instance of operation, the cooler 92 maintains the apparatus 10 at the proper temperature as sensed by sensor 88.

Thus, the apparatus of the present invention can be used to process a range of volumes of sample without compromising either cost or performance. It has the added advantage of providing improved heat removal than current state of the art devices for both static and continuous flow applications. This gives the apparatus 10 versatility in dealing with a wide range of molecules and cells that may have widely different temperature limits, sizes and shapes.

The apparatus 10 can be used for a wide range of industrial, life sciences and defense applications, including, without limitation, pharmaceuticals, cosmetics, biotechnology, agriculture, genetic engineering, gene transfer, diagnostics and even biological warfare. The apparatus of the present invention is extremely versatile in terms of the types of molecules, cells, applications and conditions amenable to successful electroporation.

Molecules that can be successfully transferred into target cells include inorganic, organic, nonnaturally occurring, naturally occurring, synthetic and biological molecules such as, without limitation, ligands, receptors, pharmacophores, imaging agents, antibodies, proteins, peptides, sugars, nucleic acids and oligonucleotides, including single-stranded and double-stranded DNA, RNA, peptide nucleic acids and chimera; nucleic acid probes, antisense, aptamer, ribozyme and chimeric constructs; allosteric molecules, labeled and tagged molecules; detectable dyes, probes, tags and markers; fluorescent and chemiluminescent molecules; enzymes, colloidal gold, dyed particles; synthetic and biological polymers and particles, including nanospheres, microspheres, beads, magnetic, paramagnetic and superparamagnetic particles, high molecular weight proteins such as ferritin and KLH, dextrans, dyed and fluorescent proteins, polymers, particles and the like. Molecules may be relatively low molecular weight organic or inorganic compounds, or they may be larger oligomers or high molecular weight macromolecules or supramolecular complexes or conjugates. They may be soluble, insoluble, colloidal, positively or negatively charged, neutral or zwitterionic. They may be spherical, rod-like, globular, symmetrical, asymmetric, chiral or achiral. Molecules of interest may be stable in a variety of aqueous or nonaqueous buffers under varying temperatures and buffer conditions. They may be produced synthetically or biologically and optionally modified, conjugated, immobilized, labeled, tagged and advantageously designed to detect, image or sense intracellular targets or events. Molecules may be introduced to target cells as a pure, homogeneous preparation, a mixture, a panel of different molecules or a diverse pool or library comprising many different molecules of known or unknown chemical composition or molecular structure.

The molecules may be tagged or encoded and may further be designed or selected for the ability to modulate the function of a target cell, or they may be specific recognition molecules capable of interrogating an intracellular structure, substance, process or event, optionally without substantially modulating or altering the function of the target cell. They may also be molecular targeting agents capable of delivering diagnostic or therapeutic agents to an intracellular structure, target or compartment.

Target cells may be, for example and without limitation, synthetic, engineered or biological cells, ghosts, microvessels or vesicles. Biological cells include transgenic, recombinant, hybrid and engineered cells. Target cells may be introduced to the sample gap either free in suspension or, as in the case of adherent cells, attached to a substrate. Biological cells may be derived from human, animal, plant, protist, fungal or moneran lineage. Cells may be electroporated under physiologic, quasi-physiologic or nonphysiologic conditions with varying cell counts, buffer conditions, additives, temperature, pH, ionic strength and the like.

The apparatus of the present invention can be used in pharmaceutical, diagnostic, agricultural, biotechnology, industrial, military and defense applications, including, for example, discovery of new drugs, diagnostic reagents and methods, library screening methods for biotechnology applications, gene transfer techniques for clinical veterinary and agricultural use, gene and cell therapies, cell-based assays for clinical and environmental biomarkers, improved bioreactor-based production and processing methods, environmental remediation and biowarfare, including defense against bioweaponry and bioterrorism.

Electroporation conditions may be conveniently varied for different applications. Pertinent optimization parameters include, for example, applied voltage and voltage gradients, pulse formats, flow rates and buffer conditions, particularly ionic strength, resistivity, composition and temperature.

Target cells and molecules to be electroporated may be introduced into the sample gap, in the alternative, by various means including pipetting, dispensing and aliquotting; robotic, automated and semi-automated means for fluid transfer; and factory incorporation of molecules, panels, libraries and/or cells in disposable or reusable cartridges.

Molecules to be transferred into cells may be added to the sample gap before or after the cells are introduced, or molecules may be mixed with cells followed by introduction of the mixture to the sample gap according to a given implementation. Molecules may comprise a pure, homogeneous preparation of a single molecular species or a plurality, panel, mixture, pool or library of different molecules. In the case of a plurality of different molecules, the different molecular species may be introduced to the sample gap simultaneously (e.g., as a single bolus addition or continuous infusion) or sequentially (e.g., by multiple or intermittent additions).

The sample may be expelled and collected by various means adaptable to continuous, discontinuous or intermittent flow, e.g., by flushing the sample gap with wash buffer, new sample or a combination of buffer and new sample. In one embodiment, cells in the expelled sample are imaged, analyzed and/or sorted according to one or more user-defined parameters (e.g., size, shape, absorbance or fluorescence) by further apparatus (not shown).

Formed of cylindrical components as shown in FIGS. 1-4 and 6, the hardware is relatively simple and can be produced economically. The design may be readily implemented in either a reusable or disposable format. Provisions can be made for disassembly and cleaning for reuse or for disposal of the chamber after use.

The apparatus 10 is of relatively small size that lends itself to be used in a multiple array (not shown) for high-volume or high-throughput applications such as pharmaceutical production, drug discovery or diagnostics, while also allowing for pilot scale or research laboratory use.

In the alternative, cells are grown and adhered to a tape (cylindrical, not shown), which is then slipped over the inner electrode shaft 14, or cells grown on a cylindrical capsule that subsequently can be removed after electroporation for experimentation or treatment. Removable, sealed plug-ins may be used as disposables in place of the shaft 14 and the fluid input pumping arrangement. In a further alternative, complete systems (not shown) for particular applications including sterile components may be packaged in disposable packaging.

In a further alternative, the electroporation chamber may include linearly moving electrode(s) which reciprocate within the chamber such as chamber 45. In this case, an actuator (not shown) is coupled to the shaft such as shaft 14 and displaces the shaft along the shaft axis 110, FIG. 6, e.g., left and right in the FIG. The amount and frequency of displacement is determined empirically for each implementation and is of relatively slow movement corresponding to the rotation of the shaft movements of FIG. 6 described above. This axis movement may also be combined with rotation movement of the shaft in a further embodiment. The embodiments described utilize the relative motion of the electrodes, i.e., movement of either electrode or both, to cause cells in suspension to move in a relatively random fashion (e.g., so as to exhibit what is termed ‘free tumbling’). Extensive formation of electropores without concomitant excessive cell damage or destruction is effected by repeatedly pulsing the sample with pulse trains configured to form a large number of electropores. A lower voltage pulse following the higher initial voltage pulses moves molecules by electrophoresis into the open pores of target cells. Effective cooling of the sample enhances cell survival.

The relative rotation or displacement of the electrodes set in a cylindrical configuration produces a relatively flat shear velocity across the radius of the sample gap, e.g., normal to the axis 110, FIG. 6, allowing cells in suspension to change orientation freely as they are pulsed. The apparatus and embodiments of the present invention provide a means to increase the statistical probability of pore formation as cells change orientation between the electrodes. Therefore, as the sample is subjected to repeated pulsing with sufficient time between pulses for the pores to reseal, each additional pulse train increases the probability of more pore formation and increased transfer of molecules into electroporated target cells.

Furthermore, since the outer cylinder forming the ground electrode is part of the cold plate of the thermoelectric cooler, excellent thermal isolation from the environment is established, and heat can be efficiently removed from the sample during electroporation. This feature is extremely important for many cells used in electroporation where temperatures must be kept below physiological levels. Whether used in the stopped batch flow or continuous process flow modes, cell loss due to I²R heating is kept to a minimum due to the thermal design as described herein.

In Marshall et al (U.S. Pat. No. 4,906,576)and Meserol U.S. Pat. No. 6,074,605, the orientation of the cells to be electroporated is considered. Marshall's method first aligns cells with a low amplitude, high frequency field pulse and then hits the aligned cells with higher voltage pulsing in a static electroporation chamber. Marshall postulates that all of the aligned cells will present the same profile to the electroporating voltages. This approach might work well with cells such as erythrocytes, but it is not valid for cells that are irregular or variable in shape or size. Certainly, it suffers from the same problem as does static electroporation, as under repeated pulsing, already opened pores might see excessive currents as they are facing the same way as when they were formed.

In one mode of operation, the apparatus according to the present invention induces suspended cells to change orientation randomly, increasing the probability that electropores will be formed on many surfaces as the tumbling cells are hit by repeated electrical pulse trains. Field analysis has shown that when the long axis of a cell is perpendicular to the electric field gradient, more electropores are formed. Therefore, when a cell is in the free tumbling mode and hit by repeated pulse trains, the probability of having the long axis presented perpendicular to the electric field at some time is increased, raising the probability of successful pore formation and electrophoresis of molecules into open pores.

The embodiments according to the present invention differ from Meserol et al. in that it is the relative motion (e.g., rotation) of the electrode(s) that induces free tumbling of suspended cells during electroporation. Furthermore, the embodiments include a unique configuration for improved heat transfer from the sample as well as thermal isolation from the environment.

The motion of the shaft 14, for example, may be produced by a prime mover including a motor, a solenoid, a pressurized piston and cylinder arrangement, and/or linkages and shaft coupled to any of these or coupled to other prime movers such as other electric field or pressurized fluid operated devices or vibrating sources. Also, the vibrations may be created by low frequency sound or other vibration sources. The vibrations may be induced by a vibrating cone and magnetic drive as in speaker arrangements or by pulsating pressurized fluids. The vibration amplitudes and frequencies may be controlled by a computer or other control. The vibrations may be used to create linear reciprocal motions of the shaft, for example, or coupled to linkages to create rotation.

In a further embodiment, the target cells may be optically detected with any of spectrophotometry, imaging, microscopy, laser scanning, fluorescence, flow cytometry or cell sorting.

It will occur to those of ordinary skill that still other various modifications of the disclosed embodiments may be provided. The disclosed embodiments are given by way of illustration, and not limitation. It is intended that the scope of the present invention be defined by the appended claims.

### EXAMPLE

The apparatus 10 of FIG. 3 was set up with manual filling and manual control of the rotation and electroporation parameters. Turkey erythrocytes obtained from Lampire Biological Laboratories, Inc. (Pipersville, Pa.) were chosen as the target cells and trypan blue dye from Sigma Cell Culture (St. Louis, Mo.). Trypan blue was added at a concentration of 0.005% to the electroporation buffer. This buffer contained MgCl₂—0.812 g/l, KCl—3.73 g/l and Tris-HCl—1.57 g/l. The pH was 8.0, adjusted from 5.12 with Tris base. Resistivity was measured at 271 ohm-cm. NaCl equivalency measured at 1.19 ppt, practical salinity—1.56 ppt and osmolality adjusted to 287 by addition of a solution of 5.5 g sucrose in 100 ml buffer.

The turkey cells were electroporated at the original hematocrit of 30% and also at dilutions of 1:5, 1:10 and 1:15. Voltage ranges of 100 v to 740 v were applied during electroporation testing. The voltage waveform of FIG. 8 was applied. For this example, electroporation was performed at 740 v. The rotor 14 was rotated at a rate of 0.1 revs./sec.

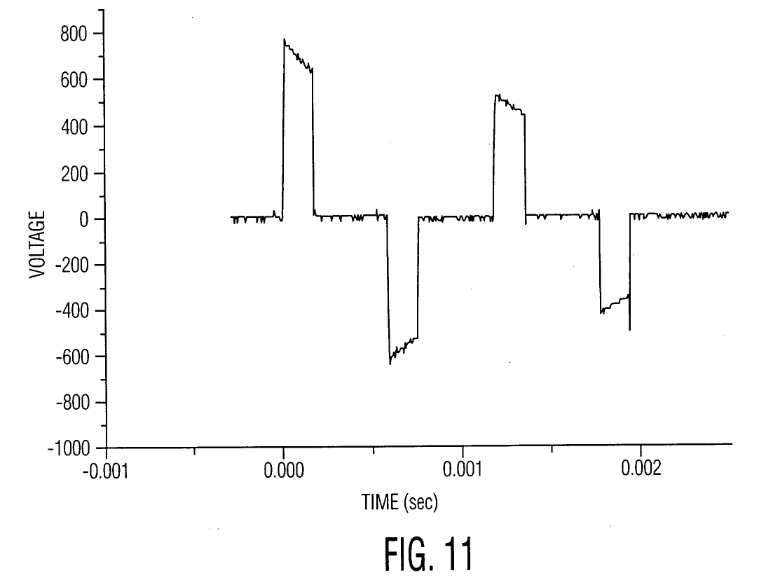

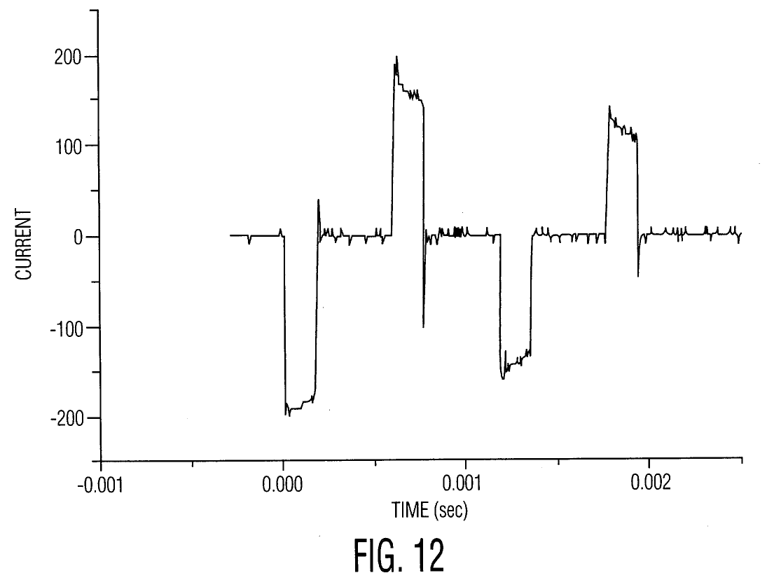

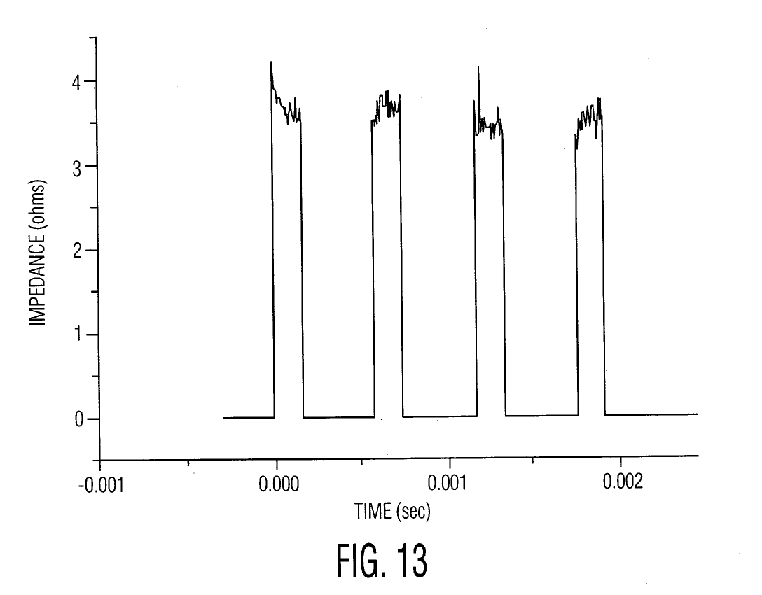

A calibration run for the original hematocrit was run at 100 v to obtain a baseline impedance for the non-electroporated cell suspension. Impedance was calculated on a dynamic basis by dividing the voltage data by the current data. FIG. 11 is the voltage waveform for the 740 v experiment. FIG. 12 is the current waveform. FIG. 13 is the impedance waveform obtained by dividing current into voltage. Note that the first impedance pulse starts out at a higher impedance than the following ones, and in fact, during the pulse, the impedance starts high and decreases. By the end of the second pulse, the impedance pulses demonstrate constant amplitude. Another way of observing this is to examine the current plot as compared to the voltage plot. The first voltage pulse decays exponentially during the ON time. Observe that the current waveform amplitude of the first pulse is essentially constant. This occurs as during the pulse, the impedance is dropping causing the effective current to remain flat as the impedance drop almost matches the voltage exponential decay.

The theory behind this behavior is that the electropores are formed virtually at the start of the first voltage pulse, probably in the first microsecond or two. Over the next two pulse periods the pores open up decreasing the solution impedance as cells with open pores act like short circuits compared with intact cells. The pulse voltage has decayed quite rapidly from the first pulse to a level below induced membrane voltage breakdown potential. Thus pore formation primarily occurs at the start of the first pulse and generally the pores complete opening up within one millisecond. All subsequent voltages act to electropherese external molecules into the cells.

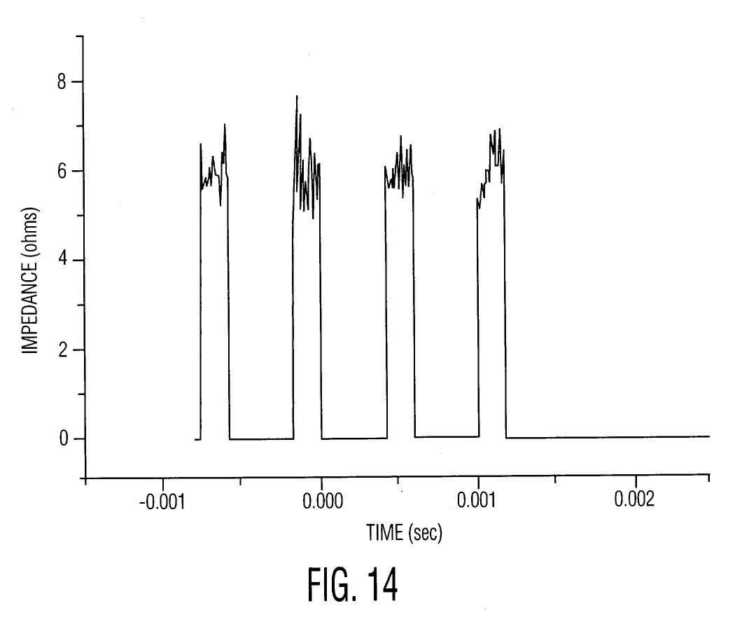

The dynamic impedance plot is a valid means of indirectly observing the electroporation of a target cell suspension. A calibration run for the impedance measurement was made by applying a 100 v pulse train to the cell suspension. (a voltage well below the membrane breakdown potential). FIG. 14 is the impedance plot for the calibration run. The average impedance was constant at a value of 5.908 ohms. The impedance plot for the 740 v experiment started at 4.14 ohms and decreased to 3.24 ohms, which is the approximate buffer impedance. The low voltage run establishes the non-electroporated impedance, while the 740 v run shows the change as the pores are formed and opened up in the target cells.

Demonstration of successful pore formation could additionally be demonstrated by microscopic examination of cells following electroporation in the presence of trypan blue or advantageously a fluorescent dye such as fluorescein, rhodamine or indocyanine green.

What is claimed:

1. An electroporation apparatus comprising:
a first electrode;
a second electrode arranged to provide a fluid receiving sample gap with the first electrode;
means for causing relative movement of the first and second electrodes;
means for applying electroporation voltage pulses across the gap; and
means for applying a fluid sample to the gap during the application of said pulses during said relative movement.

2. The electroporation apparatus of claim 1 wherein said means for causing relative movement includes at least one selected from the group consisting of a vibration source, a cylinder and piston, a solenoid and a motor.

3. The electroporation apparatus of claim 1 wherein said movement comprises at least one of rotation, translation or vibration.

4. The electroporation apparatus of claim 1 wherein the first electrode is a rotating shaft and the second electrode is a housing receiving the shaft and forming a chamber forming said gap with shaft.

5. The electroporation apparatus of claim 1 wherein the means for applying said high voltage pulses includes a switching capacitive network for creating a pulse train of alternating negative and positive first pulses.

6. The electroporation apparatus of claim 5 wherein the first pulses have a minimum amplitude of a first value and including means for limiting the amplitude of said first pulses to a given constant value lower than said first value after the expiration of a given period.

7. The electroporation apparatus of claim 1 wherein said means for applying high voltage pulses includes means for periodically applying successive spaced trains of pulses of exponentially decreasing values to said electrodes.

8. The electroporation apparatus of claim 6 wherein the first electrode is a housing forming a chamber in which said gap is disposed, the second electrode being located in the chamber forming said gap with said housing in said chamber, further including cooling means for cooling the housing.

9. The electroporation apparatus of claim of claim 8 wherein said cooling means comprises thermoelectric means.

10. The electroporation apparatus of claim 1 including means for pumping sample fluid into the gap for one of batch and continuous processing in said gap.

11. A method of electroporating a target cell comprising:
introducing the target cell into a sample gap disposed between two electrodes;
displacing at least one of the two electrodes; and
electroporating the target cell by applying a potential difference between the two electrodes during the displacing.

12. The method of claim 11 where the applying the potential difference includes applying voltage pulses of a given polarity at spaced intervals.

13. The method of claim 12 wherein the spaced intervals each have a first time value and the pulses have a pulse width of a second time value, each interval first value being at least three times the magnitude of the second value.

14. The method of claim 11 wherein the applying the potential difference includes applying a plurality of spaced voltage pulses of alternating polarity, at least one of the voltage pulses being followed by a first time period encompassing the time of occurrence of the at least one voltage pulse, no pulses being applied in the first time period.

15. The method of claim 14 including forming a network of capacitors and providing means for discharging the capacitors to produce said pulses, said means for discharging for causing said pulses to exhibit an exponential decaying amplitude from a first value, the method including reducing the voltage amplitude of the first value across the capacitors to a substantially lower second value for a second time period following the generation of said pulses of the first value.

16. The method of claim 15 including forming the second values of the pulses at a substantially constant amplitude.

17. The method of claim 11 including providing a target cell comprising at least one of a suspended cell, an adherent cell or a cell ghost.

18. The method of claim 11 further comprising introducing a molecule of interest into the sample gap.

19. The method of claim 18 includes selecting the molecule of interest from the group consisting of one or more of proteins, peptides, nucleic acids, microparticles, nanoparticles, polymers, conjugated molecules and labeled molecules.

20. The method of claim 18 including selecting the molecule of interest from the group consisting of any of a drug, a therapeutic candidate, members of a candidate library, a ligand, a receptor and an optically detectable molecule.

21. The method of claim 18 including optically detecting the target cell following the electroporation of the cell with the molecule of interest.

22. The method of claim 21 wherein the optically detecting comprises optically detecting the target cell with at least one of spectrophotometry, imaging, microscopy, laser scanning, fluorescence, flow cytometry or cell sorting.

23. The method of claim 11 wherein the applying the potential difference includes applying a first voltage pulse in a first time period position in the gap between the first electrode and the second electrode at a first polarity; and
applying a second voltage pulse in a second time period position after and spaced in time from the first position between the first electrode and the second electrode;
applying further pulses of decreasing magnitude in time periods subsequent to the second time period; and
then applying pulses of constant magnitude substantially lower in magnitude than that of the further pulses in periods following the further pulses.

24. The method of claim 23 wherein the applying of pulses in pairs, wherein each pair includes applying alternating pulses of the same magnitude and of opposite polarity for a first time period and repeating the applying of pulses for subsequent pairs of pulses in subsequent time periods with pulses of decreasing magnitude of the same polarity following each other in sequence, each pair of pulses in a subsequent time period alternating in polarity and of the same magnitude.

25. The method of claim 24 including applying pulses of constant magnitude substantially lower in value than said pairs of pulses in a period following said subsequent time period.

26. The method of claim 11 including cooling the other of said electrodes while displacing the one electrode.

27. An apparatus for electroporating a target cell comprising:
means for introducing the target cell into a sample gap disposed between two electrodes;
means for displacing at least one of the two electrodes; and
means for electroporating the target cell by applying a potential difference between the two electrodes during the displacing.

28. An apparatus for electroporating at least one cell comprising:
means for forming a fluid receiving chamber;
means for introducing a fluid containing the at least one cell into the chamber;
means for creating a shear stress in the fluid to cause the at least one cell in the chamber to change orientation; and
means for applying pulses to the means to electroporate the at least one cell.

29. The apparatus of claim 28 including means for introducing a molecule into said fluid, the electroporation for causing the molecule to engage the porated at least one cell.

30. The apparatus of claim 28 including means for thermally isolating the chamber from the ambient atmosphere while maintaining the chamber at a safe temperature for said at least one cell.

31. A method of electroporating at least one cell comprising:
forming a fluid receiving chamber;
introducing a fluid containing the at least one cell into the chamber;
creating a shear stress in the fluid to cause the at least one cell in the chamber to change orientation; and
applying pulses to the means to electroporate the at least one cell.
